(19) United States
(12) Patent Application Publication
(10) Pub. No.: US 2017/0361122 A1
(43) Pub. Date: Dec. 21, 2017

(54) IMPLANTABLE DEVICE FOR OPTICALLY STIMULATING THE BRAIN COMPRISING A MULTI-CHANNEL CATHETER

(71) Applicant: COMMISSARIAT A L'ENERGIE ATOMIQUE ET AUX ENERGIES ALTERNATIVES, Paris (FR)

(72) Inventors: Claude CHABROL, Poisat (FR); Alim-Louis BENABID, Meylan (FR)

(73) Assignee: COMMISSARIAT A L'ENERGIE ATOMIQUE ET AUX ENERGIES ALTERNATIVES, Paris (FR)

(21) Appl. No.: 15/539,295

(22) PCT Filed: Dec. 18, 2015
(86) PCT No.: PCT/EP2015/080444

§ 371 Date: Jun. 23, 2017

(30) Foreign Application Priority Data
Dec. 26, 2014 (FR) 1463353

(51) Int. Cl. A61N 5/06; A61B 18/00; A61B 18/22; A61B 17/00

(52) U.S. Cl. CPC A61N 5/0601; A61N 5/0622; A61N 5/0618; A61B 2218/002; A61B 2217/007; A61B 2017/00221; A61B 2018/00446; A61B 2018/00148; A61B 2017/00331; A61B 2017/00323; A61N 2005/0659; A61B 2018/2261

(57) ABSTRACT

An implantable device for optically stimulating a brain of a human being or animal, including: a multi-channel biocompatible catheter including a plurality of channels extending substantially parallel to each other relative to a longitudinal axis of the multi-channel catheter; a light guide, extending into one channel, for optically stimulating the brain, the multi-channel catheter acting as a sheath totally enveloping the light guide; a functional element, extending into another channel, to measure light injected into a surrounding medium at a distal end of the light guide and/or an element acting on the shape of the multi-channel catheter.

### TECHNICAL FIELD

The present invention relates to the field of deep brain stimulation of a human being or animal, and more particularly to the field of deep brain stimulation by optical irradiation.

Deep brain stimulation is a therapeutic technique including implanting a device for stimulating specific parts of the brain. Thus, different disorders, related for example to depression, Alzheimer's or Parkinson's disease can be improved. The optical deep brain irradiation implemented by the invention can in particular enable neurodegenerative diseases such as Parkinson's disease to be treated.

The invention thus provides an implantable device for optically stimulating, or even illuminating, the brain of a human being or animal, as well as an optical stimulation method implemented by means of such an implantable device and a method for implanting such an implantable device.

### STATE OF PRIOR ART

Solutions have already been provided in prior art to enable some neuron dysfunctions, including Parkinson's disease, to be treated, by optical irradiation of the brain with a light source emitting in the infrared (IR) range.

Patent application US 2009/0118800 A1 describes in particular the use of a device implantable in the brain enabling biomolecular structures of the brain, and in particular target cells having photosensitive proteins, to be optically irradiated.

Such solutions can implement the introduction of an optical fibre into the brain, through which infrared light is guided to the brain from a light source external to the brain.

However, the placement of an optical fibre into the brain of a human being or animal has many drawbacks and inconveniencies. Indeed, the insertion of the optical fibre into the brain should be made under optimum safety conditions for the patient. Since optical fibres are not generally biocompatible, it turns out to be necessary to have a sterile and sealed protection means available. Further, the implantation depth of the optical fibre, that is the length of the optical fibre between the skull and the distal end of the optical fibre, is a variable parameter which depends on the patient to be treated. Yet, the connection of the proximal end of the optical fibre to the light source requires a very accurate optical assembly which cannot be custom made at the time of the surgical operation for the case where the implantation length provided for the optical fibre would not be adapted to the patient.

### DISCLOSURE OF THE INVENTION

One purpose of the invention is to overcome at least partly the above-mentioned needs and drawbacks related to embodiments of prior art.

The invention in particular aims at providing a new type of implantable optical deep stimulation device for implanting a light guide in the brain, in particular an optical fibre, the implantation being made in a biocompatible manner by minimising the medical risks related to the use of the device, in particular risks of injuries and/or infections of an individual's body.

Thus, one object of the invention, according to one of its aspects, is an implantable device for optically stimulating the brain of a human being or animal, characterised in that it includes a biocompatible multi-channel catheter, comprising a plurality of channels extending substantially in parallel to each other relative to a longitudinal axis of the multi-channel catheter, the multi-channel catheter including a proximal end and a distal end, and in that it further includes:
a light guide, extending into a channel of the multi-channel catheter, for optically stimulating the brain, including a proximal end for receiving light emitted by a light source and a distal end for delivering this light inside the brain, the multi-channel catheter acting as a sheath for totally wrapping the light guide,
a functional element, extending into another channel of the multi-channel catheter, for measuring the light injected into the surrounding medium at the distal end of the light guide, and/or an element acting on the form of said multi-channel catheter.

By virtue of the invention, the implantation of a light guide, in particular an optical fibre, into the brain of a human being or animal can be made in a simple, localised and secure way using the implantable device. The implantable device can enable an illumination of the human or animal brain to be made, for example in the near infrared, while minimising the medical risks during the implantation and ensuring esthetical and physical comfort for the individual, the light guide forming a barrier to physiological liquids. It can be used for illuminating brain tissues with different purposes according to the intended application, such as neuro-protection, opto-genetics, stimulation, among other things.

By “element acting on the form of said multi-channel catheter”, it is meant for example a rigid element, or stiffener, able to be introduced into said other channel, so as to induce rigidification of the channel. It can also be an element disposed in said other channel, so as to enable said multi-channel catheter to be deformed about a balance position, and then to come back to said balance position, said balance position being for example bent.

The implantable device according to the invention can further include one or more of the following characteristics taken alone or according to any technically possible combinations.

Advantageously, the multi-channel catheter is flexible such that it enables, among other things, any trauma at the individual's tissues to be avoided. The multi-channel catheter can thus be advantageously folded as need be, in particular be bent. It can be for example made of silicon or polyurethane (PU). It can be transparent.

The multi-channel catheter is advantageously adapted to its implantation in the third ventricle by passing through one of the lateral ventricles.

Furthermore, the multi-channel catheter can be thin, having in particular a cylindrical shape with a diameter between 1 and 2.2 mm. The thinness of the multi-channel catheter is in particular sufficient to enable it to be inserted into the third ventricle or in contact with any other brain zone surgically accessible through a trepanation operation of a few millimetres. The “trepanation operation” refers to the common operation made by a neurosurgeon when implanting a ventricular catheter, for example for treating hydrocephalus or placing deep stimulation electrodes for treating trauma in the case of Parkinson's disease.

As previously set out, the multi-channel catheter forms a sheath for totally wrapping the light guide. Still in other words, the light guide can be wholly overmoulded by the multi-channel catheter (full coating), such that it is possible to avoid any leak of physiological liquids, in particular blood.

Preferentially, the multi-channel catheter of the implantable device according to the invention can be performed to be suited to the individual's anatomy, in particular to the anatomy of the ventricles.

The light guide consists for example of a flexible longitudinal light guide, for example an optical fibre.

On the other hand, advantageously, the functional element, in particular the monitoring probe makes it possible to ensure that the device properly works and to check the light dose applied to the individual. The measurement of the light injected in the surrounding medium enables the measurement of the light injected in the individual's tissues to be known.

Further, the distal end of the multi-channel catheter can be of an oblong shape.

Advantageously, an oblong shape of the distal end of the multi-channel catheter can enable the penetration into the individual's tissues to be facilitated.

The distal end of the multi-channel catheter can on the other hand include a light scattering element, the light scattering element being in particular located at the distal end of the light guide inside the channel into which the light guide extends.

The scattering element can be adapted in length to the area to be treated of the brain by optical stimulation. The scattering element can in particular extend into the multi-channel catheter, and in particular into the channel of the multi-channel catheter in which the light guide is located, over a length between 2 and 20 mm, for example in the order of 10 mm, as a function of the intended application. Thereby, it is possible to obtain a linear scattering source enabling the lighting uniformity of a great brain area to be improved, in particular of the third ventricle and the Substantia Nigra pars compacta.

Advantageously, the presence of this scattering element enables a lens effect at the end of the preferentially transparent multi-channel catheter to be avoided. The scattering element enables in particular the risks of too high a power density to be limited. The scattering element can in particular include a titanium dioxide (TᵢO₂) load to be included.

The scattering element can in particular enable radiation from the light guide to be scattered, before it reaches the tissues/cells of the brain. A light source generating a relatively significant power area density can be used, without risk of damage to the tissues/cells of the brain.

Furthermore, the light scattering element can include a fluorophore for fluorescence monitoring.

Such a fluorophore can for example consist of the IndoCyanine Green (ICG) pigment, with absorption wavelengths between 600 and 900 nm and emission wavelengths between 750 and 950 nm.

Further, the multi-channel catheter can include a fluid channel for injecting and/or sampling liquids, in particular for injecting a contrast agent during the surgical phase.

The presence of such a fluid channel can enable viewing of the ventricles to be facilitated without requiring another trepanation.

Such a fluid channel can in particular enable any other product necessary to the treatment of the individual to be injected. The distal end of such a fluid channel is advantageously provided with a hole or a slot to enable liquids to be injected and/or sampled.

Furthermore, the multi-channel catheter can include at least one channel for using a stiffener during the placement of the multi-channel catheter.

The multi-channel catheter can in particular include at least one channel for using a localised stiffener of super elastic material or using the ability of a thermoplastic waveguide to be custom thermoformed on the finished device.

Such a stiffener can in particular correspond to Nitinol from 250 μm to 500 μm. It can be in the form of a shape memory rod.

Furthermore, the multi-channel catheter can include a channel equipped with a radiopaque label for post-surgical check.

The presence of such a radiopaque label is advantageously required to facilitate checking that the multi-channel catheter is properly positioned.

Such a radiopaque label can for example include an edge of a barium sulphate (BaSO₄) load. Such a radiopaque, non-transparent, edge can be disrupted at the possible scatterer if the same is not directional.

The multi-channel catheter can also include at least one channel for passing conductive electrodes for electrically stimulating the brain at the distal end of the multi-channel catheter.

The multi-channel catheter can also include another channel for passing another light guide for recovering light during monitoring performed via the functional element.

On the other hand, the multi-channel catheter can include an anti-crushing anti-folding protective means, the multi-channel catheter including in particular at least one bent portion and the anti-crushing anti-folding protective means being located at said at least one bent portion.

Advantageously, the anti-crushing anti-folding protective means enables rupture of the light guide to be avoided during the surgical operation. It can be integrated inside the multi-channel catheter or located externally to the same and fastened via attachments.

The light guide can be made of different transparent materials having a suitable index, such as silica, silicon or thermoplastics, such as polymethyl methacrylate (PMMA). It can also include at least one bent portion shaped by localised heating. Such a shaping can be made during surgery depending on the angle desired by the surgeon thanks to a tooling locally heating the light guide through the multi-channel catheter, made in particular of silicon. A moulding temperature of about 70° C. can be used, much lower than silicon degradation temperatures, higher than 200° C.

The implantable device according to the invention can further include a plurality of connecting elements at the proximal end of the multi-channel catheter, and in particular at least one optical connecting element for connecting at least one light guide to the light source, a fluidic connecting element for injecting products during the surgical phase, in particular a contrast agent, and/or for connecting to an implantable delivery pump for using photosensitive products, an electrical connecting element for applying an electric field in the illuminated zone and/or performing an electrical measurement.

Further, the multi-channel catheter can include a metal coating, in particular on the wall of at least one of its channels, and in particular the channel including the light guide, for promoting a light emitting angle and/or performing a selective scattering.

The implantable device according to the invention can also include a light source, emitting in particular in the infrared range, connected to at least the proximal end of the light guide.

The light source can be integrated to a neurostimulator, in particular a deep brain stimulation (DBS) type neurostimulator.

Alternatively, the light source can be independent of a neurostimulator.

Still in other words, the light source can be offset from a neurostimulator. Advantageously, the use of an offset light source can enable commercial neurostimulators to be used, or even the use on individuals already equipped with DBS type probes. This solution can in particular allow neuron protection to stop neuron degeneration simultaneously to a deep electrical stimulation to compensate for the lack of dopamine in individuals with an advanced stage of the disease.

The light source can be located inside a sealed biocompatible casing coupled to at least one light guide, in particular via a removable connector.

The casing can include an optical detector for monitoring light injection coupled to the functional element and electronic wireless communication means with a remote terminal for checking the device.

On the other hand, the implantable device according to the invention can further include a power source, in particular a continuous, modulated or pulsed source, for example a storage battery or a battery cell, for powering the light source.

Further, the implantable device according to the invention can also be remotely powered via an external power antenna.

The light source can also include a sensor and a dichroic mirror, located between the sensor and the scattering element, the sensor enabling wavelengths back from the scattering element emitted by fluorescence to be measured.

The light source can in particular emit in the near infrared range. The light source can in particular be arranged such that it emits light with wavelengths preferentially between 650 nm and 950 nm, for example in the order of 670 nm.

The light source can be intended to be implanted in the sub-cutaneous route, that is under the scalp.

The light source can consist of any light source capable of emitting in the infrared, such as a laser diode, a Light-Emitting Diode (LED) or a Vertical-Cavity Surface-Emitting Laser diode (VCSEL), for example. Preferentially, the light source includes a laser diode.

Another object of the invention is, according to another of its aspects, a method for optically stimulating the brain of a human being or animal implemented by means of an implantable device as previously defined, wherein:

(a) light in the infrared range, and in particular in the near infrared range, is emitted,

(b) this light is transmitted to a proximal end of a light guide implanted in the brain of the human being or animal,

(c) this light is guided in the light guide up to a distal end of the light guide such that this light irradiates the inside of the brain from its distal end.

Another object of the invention is, according to another of its aspects, a method for implanting an implantable device as previously defined for optically stimulating the brain of a human being or animal, wherein a trepanation operation is made for implanting the multi-channel catheter in the brain of the human being or animal.

The optical stimulation method and/or implantation method according to the invention can further include at least one the following steps:
the distal end of the light guide is implanted in proximity of the Substantia Nigra pars compacta (SNc),
the distal end of the light guide is implanted in the third ventricle of the brain, which is located in proximity of the substantia nigra pars compacta,
the distal end of the light guide is implanted in proximity of, or even in contact with, the floor of the third ventricle of the brain.

The optical stimulation method and the implantation method according to the invention can include any of the previously set out characteristics, taken alone or according to any technically possible combinations with other characteristics.

### BRIEF DESCRIPTION OF THE DRAWINGS

The invention will be better understood upon reading the detailed description that follows, of exemplary implementations not limiting the same, as well as upon examining the schematic and partial figures of the appended drawing, in which:

FIG. 1 partially illustrates an exemplary embodiment of an implantable device in accordance with the invention,

FIG. 2 partially illustrates, in a cross-section and perspective view, an exemplary embodiment of a multi-channel catheter of another implantable device in accordance with the invention, and

FIGS. 3 and 4 represent two other exemplary implantable devices in accordance with the invention, respectively including a light source integrated to a neurostimulator and a light source offset relative to a neurostimulator.

Throughout the figures, identical references can designate identical or analogous elements.

Moreover, the different parts represented in the figures are not necessarily drawn at a uniform scale, to make the figures more legible.

### DETAILED DISCLOSURE OF PARTICULAR EMBODIMENTS

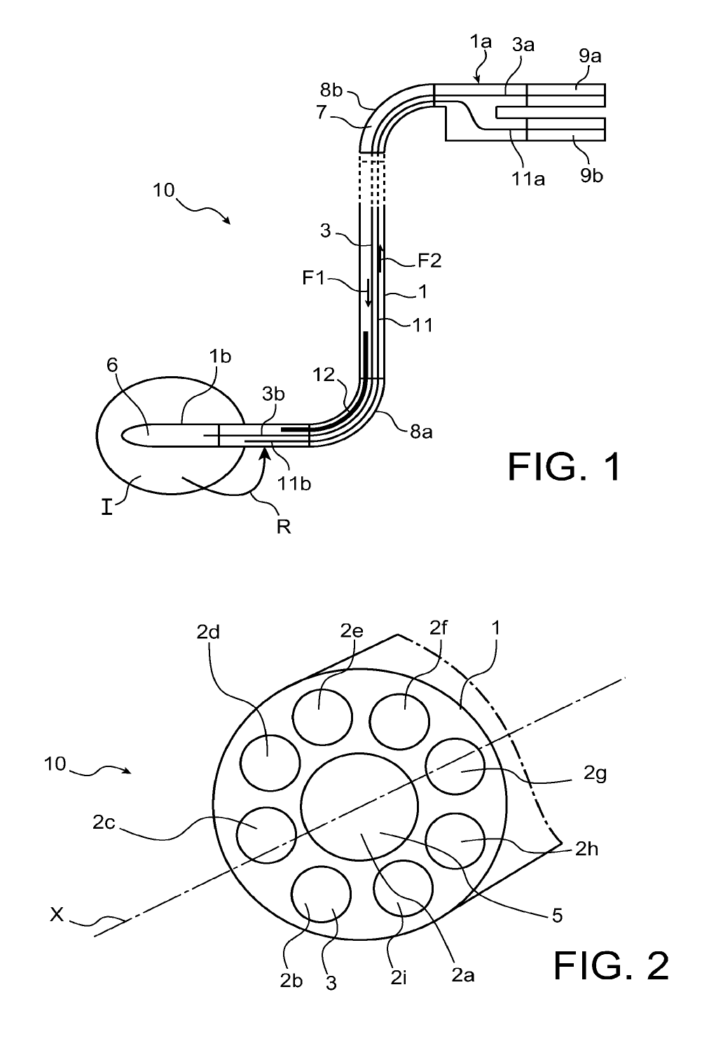

In FIG. 1, an exemplary implantable device 10 in accordance with the invention for optically stimulating the brain of a human being or animal is partially illustrated.

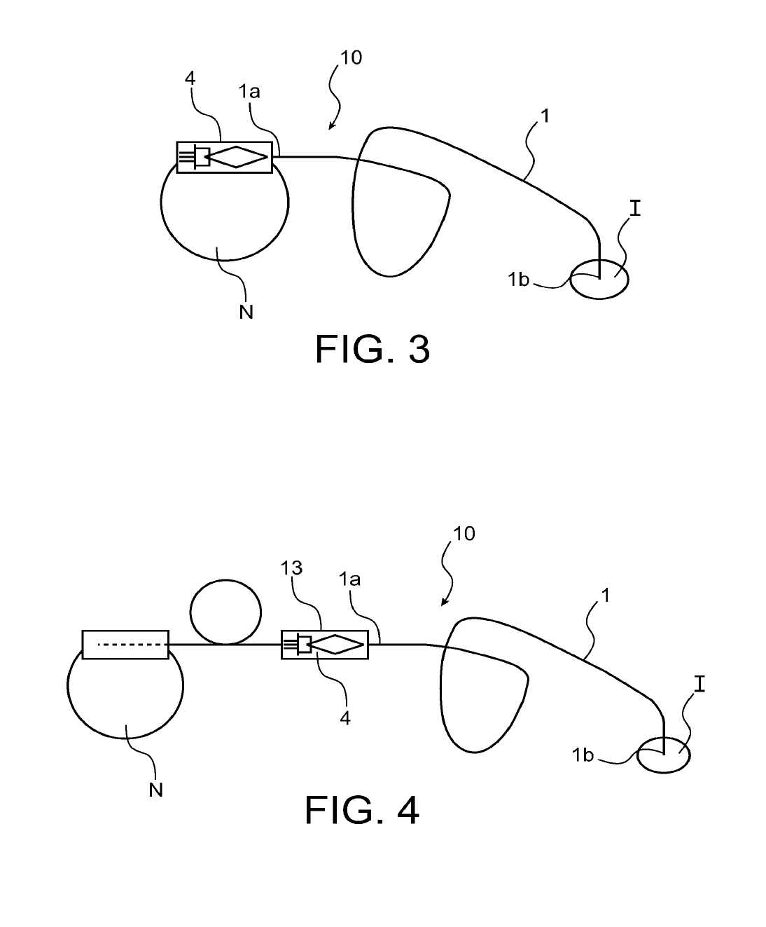

The implantable device 10 includes a multi-channel catheter 1 in the form of a tube, provided in this example with two bent portions 8a and 8b, a proximal end 1a, intended to be connected to a light source 4 (visible in FIGS. 3 and 4) and possibly other optical and/or electronic systems, and with a distal end 1b at which a light guide 3 emits illumination I towards the human or animal brain.

The multi-channel catheter 1 is preferentially made of silicon or polyurethane (PU), and is in a transparent form. It has a cylindrical shape with a diameter between 1 and 2.2 mm, and its thinness is such that it enables it to be inserted into the third ventricle or in contact with any other zone of the brain surgically accessible through a trepanation operation of a few millimetres.

Moreover, in order to facilitate penetration into tissues, the distal end 1b of the multi-channel catheter 1 can be of an oblong shape, in particular for catheters having a diameter higher than 1.3 mm.

The multi-channel catheter 1 is advantageously biocompatible, and comprises a plurality of channels 2a-2i (visible in FIG. 2) which extend in parallel to each other relative to the longitudinal axis X of the catheter 1. Still in other words, on each rectilinear portion of the multi-channel catheter 1, the channels 2a-2i are parallel to each other.

The light guide 3, in particular in the form of an optical fibre or a high index contrast waveguide injected into the channel, extends in a first channel 2b of the catheter 1. It enables the human or animal brain to be optically stimulated.

The light guide 3 includes a proximal end 3a which receives light emitted by the light source 4 and the distal end 3b for delivering this light inside the brain as an illumination I.

Advantageously, the multi-channel catheter 1 forms a full protective coating for the light guide 3.

On the other hand, the distal end 3b of the light guide 3 includes a light scattering element 6. This scattering element 6 extends for example into the first channel 2b of the multi-channel catheter 1 in which the light guide 3 is located over a length in the order of 10 mm. Thereby, it is possible to obtain a linear scattering source enabling the lighting uniformity of a great brain area to be improved, and also the lens effect to be avoided at the end of the multi-channel catheter 1.

This scattering element 6 includes in particular a titanium dioxide (TᵢO₂) load. Moreover, this scattering element 6 can include a fluorophore for fluorescence monitoring, consisting for example of the IndoCyanine Green (ICG) pigment.

On the other hand, in FIG. 1, it is also noticed that the implantable device 1 can include a plurality of connecting elements 9a, 9b at the proximal end 1a of the multi-channel catheter 1.

In particular, the multi-channel catheter 1 includes an optical connecting element 9a for connecting the light guide 3 to the light source 4.

Moreover, the multi-channel catheter 1 also includes another optical connecting element 9b which enables another light guide 11, which is provided with a proximal end 11a and a distal end 11b, to be connected to the light source 4. Both optical connecting elements or pedestals 9a and 9b can be integrated if need be into a single connection module compatible with a suitable optical transceiving module.

Although not represented, the implantable device 10 can also include a fluid connecting element for injecting products during the surgical phase, in particular a contrast agent and/or for connecting to an implantable delivery pump for using photosensitive products, an electrical connecting element for applying an electric field in the illuminated zone and/or performing an electrical measurement.

On the other hand, the first bent portion 8a can be rigidified via a shape memory rod of super elastic material 12, corresponding for example to Nitinol from 250 μm to 500 μm.

Further, the second bent portion 8b includes an anti-crushing anti-folding protective means 7 so as to prevent light guides 3 and 11 from being ruptured during the surgical operation. This anti-crushing device can for example be made of a spiral yarn with implantable stainless steel as a material. The length of the anti-crushing zone can be adapted depending on the length implanted into the brain so as to protect the bend at 90° at the outlet of the cranium. This protective means 7 can be integrated into the multi-channel catheter 1 or located externally to the same and fastened via attachments.

On the other hand, in FIG. 2, an exemplary embodiment of a multi-channel catheter 1 of another implantable device 10 in accordance with the invention has been illustrated in a cross-section perspective view.

In this example, it is noticed that the multi-channel catheter 1 includes nine channels parallel to each other, including a first channel 2b in which the light guide 3 is located.

Further, the multi-channel catheter 1 also includes a central channel 2a about which the other eight side channels 2b-2i are distributed.

Therefore, the central channel 2a includes, in accordance with the invention, a monitoring probe 5, which extends into the central channel 2a, for measuring light injected into the surrounding medium at the distal end 3b of the light guide 3. Advantageously, the monitoring probe makes it possible to ensure that the device 10 is properly working and to check the light dose applied to the individual. The measurement of the light injected in a surrounding medium enables the measurement of light injected in the individual's tissues to be known.

On the other hand, as can be seen in FIG. 2, the multi-channel catheter 1 also includes a fluid channel 2e for injecting and/or sampling liquids, and in particular for injecting a contrast agent during the surgical phase, the distal end of which is pierced to enable liquids to be injected and/or sampled.

Moreover, the multi-channel catheter 1 also includes two channels 2d and 2h for using a stiffener during the placement of the multi-channel catheter 1. Such a stiffener is in particular a localised stiffener of super elastic material of the 250 μm Nitinol type.

In order to facilitate checking that the multi-channel catheter 1 is properly positioned, the same also includes a channel 2f equipped with a radiopaque label for post-surgical check. This radiopaque label can for example include an edge of a sulphate barium (BaSO₄) load.

Further, the multi-channel catheter 1 includes two channels 2g and 2i for passing conductive electrodes for electrically stimulating the brain at the distal end 1b of the multi-channel catheter 1.

It also includes a channel 2c for passing another light guide 11 for recovering light upon monitoring performed via the monitoring probe 5. Indeed, as depicted in FIG. 1, when the first light guide 3 sends light along the arrow F1 towards the scattering element 6 for illuminating I the brain, a recovered part R can be directed to the second light guide 11 which sends it back along the arrow F2 to the light source 4.

On the other hand, as previously indicated, the implantable device 10 advantageously includes a light source 4, emitting in the infrared range, connected to the proximal ends 3a and 11a of the first 3 and second 11 light guides.

FIGS. 3 and 4 thus represent two other exemplary implantable devices 10 in accordance with the invention, respectively including a light source 4 integrated to a neurostimulator N and a light source 4 offset with respect to a neurostimulator N.

In the example of FIG. 3, the light source 4 is integrated to the neurostimulator N, this being in particular of the deep brain stimulation (DBS) type.

On contrast, in the example of FIG. 4, the light source 4 is independent of the neurostimulator N, that is it is offset from the neurostimulator N.

Advantageously, the use of an offset light source 4 can enable commercial neurostimulators to be used, or even the use on individuals already equipped with DBS type probes.

On the other hand, the light source 4, in the example of FIG. 4, is located inside a biocompatible sealed casing 13 coupled to at least one light guide 3, in particular via a removable connector.

This casing 13 can include an optical detector for monitoring injecting light coupled to the monitoring probe 5 and electronic wireless communication means with a remote terminal for checking the device 1.

The implantable device according to the invention can advantageously enable deep brain illumination to be made on a human being or animal, while being able to be associated with other stimulation modes, such as electrical stimulation or product injection stimulation.

Of course, the invention is not limited to the exemplary embodiments just described. Various modifications can be made thereto by those skilled in the art.

The phrase “including one” should be understood as being synonym of “including at least one”, unless otherwise specified.

1-19. (canceled)

20: An implantable device for optically stimulating a brain of a human being or animal, comprising:
a biocompatible multi-channel catheter, comprising a plurality of channels extending in parallel to each other relative to a longitudinal axis of the multi-channel catheter, the multi-channel catheter including a proximal end and a distal end;
a light guide, extending into a channel of the multi-channel catheter, for optically stimulating the brain, including a proximal end for receiving light emitted by a light source and a distal end for delivering the light inside the brain, the multi-channel catheter acting as a sheath for totally wrapping the light guide;
a functional element, extending into another channel of the multi-channel catheter, for measuring light injected into a surrounding medium at the distal end of the light guide, and/or an element acting on a form of the multi-channel catheter;
the distal end of the multi-channel catheter including a light scattering element including a fluorophore for fluorescence monitoring.

21: The device according to claim 20, wherein the distal end of the multi-channel catheter is of an oblong shape.

22: The device according to claim 20, wherein the light scattering element is located at the distal end of the light guide inside the channel into which the light guide extends.

23: The device according to claim 20, wherein the multi-channel catheter includes a fluid channel for injecting and/or sampling liquids.

24: The device according to claim 20, wherein the multi-channel catheter includes at least one channel for using a stiffener during placement of the multi-channel catheter.

25: The device according to claim 20, wherein the multi-channel catheter includes at least one channel for using a localized stiffener of super elastic material or using ability of a thermoplastic waveguide to be custom thermoformed on the finished device.

26: The device according to claim 20, wherein the multi-channel catheter includes a channel including a radiopaque label for post-surgical check.

27: The device according to claim 20, wherein the multi-channel catheter includes at least one channel for passing conductive electrodes for electrically stimulating the brain at the distal end of the multi-channel catheter.

28: The device according to claim 20, wherein the multi-channel catheter includes another channel for passing another light guide for recovering light during monitoring performed via the functional element.

29: The device according to claim 20, wherein the multi-channel catheter includes an anti-crushing anti-folding protective means, the multi-channel catheter including at least one bent portion and the anti-crushing anti-folding protective means being located at the at least one bent portion.

30: The device according to claim 20, comprising a plurality of connecting elements at the proximal end of the multi-channel catheter, a fluidic connecting element for injecting products during a surgical phase, an electrical connecting element for applying an electric field in an illuminated zone and/or performing an electrical measurement.

31: The device according to claim 20, wherein the multi-channel catheter includes a metal coating, for promoting a light emitting angle and/or performing a selective scattering.

32: The device according to claim 20, further comprising a light source connected to at least the proximal end of the light guide.

33: The device according claim 32, wherein the light source is integrated to a neurostimulator.

34: The device according to claim 32, wherein the light source is independent of a neurostimulator.

35: The device according to claim 34, wherein the light source is located inside a sealed biocompatible casing coupled to at least one light guide.

36: The device according to claim 35, wherein the casing includes an optical detector for monitoring light injection coupled to the functional element and electronic wireless communication means with a remote terminal for checking the device.

37: The device according to claims 32, further comprising a power source for powering the light source.

38: The device according to claim 32, wherein the light source includes a sensor and a dichroic mirror, located between the sensor and the scattering element, the sensor enabling wavelengths back from the scattering element emitted by fluorescence to be measured.
